(19) United States
(12) Patent Application Publication
(10) Pub. No.: US 2023/0375506 A1
(43) Pub. Date: Nov. 23, 2023

(54) SENSOR FOR MEASUREMENT OF RADICALS

(71) Applicant: Applied Materials, Inc., Santa Clara, CA (US)

(72) Inventors: Mehran Moalem, Fremont, CA (US); Mehdi Balooch, Berkeley, CA (US)

(21) Appl. No.: 18/198,123

(22) Filed: May 16, 2023

Related U.S. Application Data
(60) Provisional application No. 63/343,502, filed on May 18, 2022.

(51) Int. Cl. G01N 29/02; G01N 33/00; H01J 37/32

(52) U.S. Cl. CPC G01N 29/022; G01N 33/0027; H01J 37/32981; G01N 2291/014; G01N 2291/021; H01J 37/32357; H01J 2237/334

(57) ABSTRACT

A sensor device comprises a quartz crystal microbalance (QCM) and a coating on at least a portion of a surface of the QCM, wherein the coating selectively reacts with radicals of a target gas and does not react with stable molecules of the target gas. The QCM is configured such that a resonant frequency of the QCM changes in response to reaction of the radicals of the target gas with the coating, wherein the change in the resonant frequency of the QCM correlates to an amount of the radicals of the target gas that have reacted with the coating.

### RELATED APPLICATIONS

This patent application claims the benefit under 35 U.S.C. § 119(e) of U.S. Provisional application No. 63/343,502, filed May 18, 2022.

### TECHNICAL FIELD

The instant specification relates to gas measurements, and in particular to measurement of radicals and/or ions in a gas flow.

### BACKGROUND

Many processes, such as processes for forming semiconductors, photovoltaics, displays, etc., use one or more gases to deposit layers, etch layers, clean substrates, and so on. For some processes a plasma is formed and used during deposition, etching, cleaning, etc. Traditionally, a flow sensor such as a mass flow controller is used to detect the amount of gas that is flowed. However, current sensors are unable to measure specific subspecies of a gas, such as just the amount of radicals in a gas, or just the amount of ions in the gas.

In semiconductor processing, radical species are often used for various processing operations in a chamber. For example, a radical species, such as atomic fluorine, may be used in an etching or a chamber cleaning process. Radical species can be formed by various processes. One process to generate radical species is to use a plasma. For example, a fluorine containing gas is flown into the chamber, and the plasma breaks the compound into elemental fluorine. Radical species are highly chemically reactive.

Process control of radical species is difficult. Particularly, it is currently not possible to effectively measure radical species concentration in a processing chamber. This is due, in part, to the highly reactive nature of the radical species. The radical species react whenever the radical species contacts any surface or other compound. Even if the surface does not react with the radical species, it still may serve as a site for recombination of the radicals with each other thus converting the species to other useless compounds. As such, existing mass spectrometry tools are not able to measure the concentration of radical species. Without the ability to quantitatively measure the radical species concentrations, effective process control, such as closed loop control, is not possible in existing semiconductor-manufacturing tools.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular implementations of the disclosure or any scope of the claims. Its sole purpose is to present some concepts of the disclosure in a simplified form as a prelude to the more detailed description that is presented later.

In one aspect of the disclosure, a sensor device comprises a quartz crystal microbalance (QCM) and a coating on at least a portion of a surface of the QCM, wherein the coating selectively reacts with radicals of a target gas and does not react to stable molecules of the target gas. The QCM is configured such that a resonant frequency of the QCM changes in response to reaction of the radicals of the target gas to the coating, and wherein the change in the resonant frequency of the QCM correlates to an amount of the radicals of the target gas that have reacted with the coating.

In one aspect of the disclosure, a manufacturing system comprises a plasma source to generate a plasma, a process chamber connected to the plasma source via one or more delivery lines, and a sensor device connected to at least one of the plasma source, the process chamber or the one or more delivery lines. The sensor device comprises a quartz crystal microbalance (QCM) comprising a coating that selectively reacts with radicals of a target gas and does not react to stable molecules of the target gas to measure an amount of the radicals of the target gas.

In one aspect of the disclosure, a method comprises receiving a gas flow comprising one or more gases, the one or more gases comprising a first plurality of stable molecules of a target gas and a second plurality of radicals of the target gas. The method further comprises measuring the second plurality of radicals of the target gas without measuring the first plurality of stable molecules of the target gas using a quartz crystal microbalance (QCM) comprising a coating on at least one surface that reacts with the second plurality radicals of the target gas but not with the first plurality of stable molecules of the target gas.

### BRIEF DESCRIPTION OF THE DRAWINGS

The present disclosure is illustrated by way of example, and not by way of limitation in the figures of the accompanying drawings.

FIG. 1 is a sectional side view of a substrate processing system including a radical sensor, according to some embodiments.

FIG. 2A is a sectional side view of a radical sensor, according to some embodiments.

FIG. 2B is a back view of the radical sensor of FIG. 2A, according to some embodiments.

FIG. 2C is a front view of the radical sensor of FIG. 2A, according to some embodiments.

FIG. 2D is a sectional side view of the radical sensor of FIG. 2A with the addition of a charged grating or grid, according to some embodiments.

FIG. 2E shows an equivalent circuit for a piezoelectric resonator, according to some embodiments.

FIG. 2F shows an exploded view of one example of a radical sensor included in a sensor holder, according to some embodiments.

FIG. 2G shows an example sensor holder for a radical sensor, in accordance with some embodiments.

FIG. 2H shows an example sensor holder for a radical sensor, in accordance with some embodiments.

FIG. 3 is a flow chart of one embodiment of a method for manufacturing a radical sensor.

FIG. 4 is a flow chart of one embodiment for a method of controlling a plasma source using a radical sensor.

### DETAILED DESCRIPTION

Embodiments of the present disclosure relate to a new type of sensor that can detect specific species of molecules and/or atoms, such as radicals and/or ions of specific gases. Conventional gas sensors (e.g., such as those in mass flow controllers) measure a total amount of gases, and cannot distinguish between specific species of molecules and/or atoms of gases. For example, a mass flow controller can measure a total gas flow, but cannot measure an amount of any specific type of gas or an amount of a specific molecular species of a particular type of gas. Embodiments discussed herein provide a sensor device that can detect amounts and/or concentrations of specific molecular species of specific gases. For example, sensor devices discussed in embodiments herein may be designed to measure an amount of fluorine radicals, or an amount of hydrogen radicals, or an amount of nitrogen radicals, which sensor devices have heretofore been incapable of detecting without use of expensive optical equipment such as spectroscopy equipment.

Embodiments include sensor devices that employ specialized coatings on surfaces of piezoelectric materials that oscillate at measureable resonant frequencies. The coating acts as a filter that filters out all molecules except for radicals of a target gas species. An example of such a piezoelectric material that may be used is quartz. For example, embodiments include a quartz crystal microbalance (QCM) with such a specialized coating on one surface of the QCM. The specialized coatings are designed for specific applications, and are reactive to only select molecular gas species used in those specific applications. Examples of applications that the sensor devices may be designed for include etch operations, plasma assisted deposition processes (e.g., plasma assisted atomic layer deposition), and so on. The coating on the piezoelectric material changes mass based on a reaction of the coating to the select molecular gas species (e.g., to radicals of a particular molecule). The change in the coating's mass causes the resonant frequency at which the piezoelectric material oscillates to change. This change in the resonant frequency is measureable, and may be used to determine the quantity of the molecular species that reacted with the coating. Accordingly, the sensor devices can directly measure specific molecular species of particular gases (e.g., fluorine radicals, hydrogen radicals, etc.). Such direct measurement of radicals enables closed loop control of plasma sources.

In an example, for a fluorine-based etch process, an etch rate may strongly correlate to a concentration of fluorine radicals. However, heretofore the concentration of fluorine radicals has not been directly detectable, and so engineers would guess at the concentration of fluorine radicals based on other known values such as a known plasma power, a known gas flow rate, and so on. By using a sensor device as described herein the amount of fluorine radicals being flowed may be directly measured, and this measurement may be used to finely control the amount of radicals being output by a plasma source, such as a remote plasma source (RPS).

Without the ability to have a quantitative measurement of the concentration of radical species, closed loop control of the processing environment is not possible. Closed loop control refers to the use of quantitative measurements as a feedback signal to a controller in order to modify processing conditions in an ongoing process. For example, in the case of the measurement of radical species, a concentration of the radical species can be measured, and the measured value can be compared to a setpoint value. When the measured value is below the setpoint value, processing parameters may be changed to increase the generation rate and output concentration of radical species, or when the measured value is above the setpoint value, processing parameters may be changed to decrease the concentration of radical species. As such, more stable and reproducible processes can be implemented in embodiments. Accordingly, embodiments disclosed herein include a radical sensor that includes of a piezoelectric oscillator (e.g., a QCM) having a surface that is coated with a film that is reactive to a target radical species of a target gas or molecule, but that is not reactive to stable molecules of the gas or molecule or to radical or stable species of other gases or molecules that are flowed together with the target gas or molecule. The radical sensor may be used for closed loop control of plasma sources.

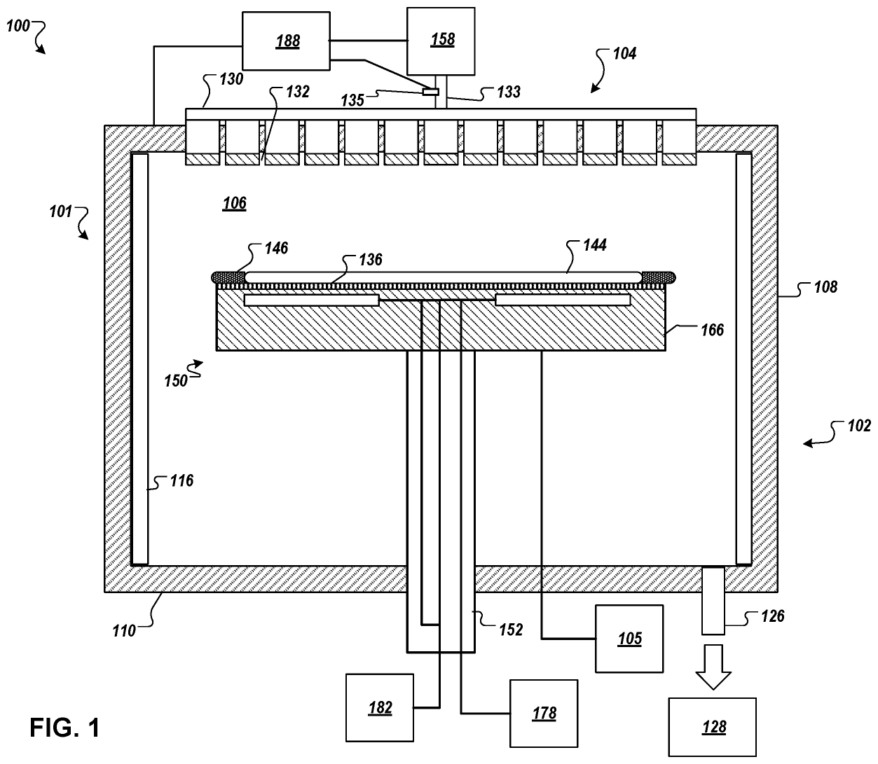

FIG. 1 is a sectional view of a manufacturing system 100 that performs plasma-based processes in embodiments. The manufacturing system may include a processing chamber 101 coupled to a plasma source 158 via one or more gas delivery lines 133. The processing chamber 101 may be, for example, a plasma etch reactor, a deposition chamber, etc. The processing chamber may be suitable for an etching operation, a deposition operation, a chamber cleaning operation, a plasma treatment operation, or any other type of operation typical of a semiconductor manufacturing facility. In an embodiment, one or more substrates (e.g., wafers) 144 may be provided within the processing chamber 101. In an embodiment, processing chamber 101 may be maintained at a pressure suitable for the target operation. In a particular embodiment, the pressure may be between approximately a fraction of a Torr and approximately 200 Torr.

The processing chamber 101 and/or plasma source 158 may be connected to a controller 188, which may control processing of the plasma source 158 and/or processing chamber 101 (e.g., by controlling set points, loading recipes, and so on). A radical sensor 135 may be connected to the gas delivery line(s) 133 to detect a concentration of radicals in a gas or plasma delivered by the plasma source 158.

In an embodiment, the manufacturing system 100 may comprise a radical sensor 135 that is fluidically coupled to the processing chamber 101 and/or to the gas delivery line(s) 133. For example, a valve may be provided along a tube between the processing chamber 101 and the radical sensor 135. In an embodiment, the valve is a type of valve that allows for an unobstructed line of sight between the processing chamber 101 and the radical sensor 135. For example, the valve may be an isolation gate valve. An isolation gate valve may allow for a binary state of operation. That is, the valve may be open (i.e., 1) or closed (i.e., 0). When the valve is open, the line of sight is unobstructed. Alternately, another type of valve such as a needle valve may be used.

In embodiments, the radical sensor 135 comprises a piezoelectric substrate in a holder. The piezoelectric substrate is made to oscillate at a resonant frequency by applying an alternating current to the piezoelectric substrate. One or more surface of the piezoelectric substrate is coated by a film that is reactive to a narrow range of molecular species. In particular, the film is composed of a material that is reactive to a target molecular species of a particular target gas from among gases being used in a process. In one embodiment, the radical sensor comprises a QCM having at least one coated surface that is coated with a film that is selectively reactive to radicals of a particular gas. The radical sensor 135 is described in greater detail below with reference to the proceeding figures.

In embodiments, the plasma source 158 is a remote plasma source (RPS) that generates plasma at a remote location and delivers the externally generated plasma to the processing chamber 101. Alternatively, the processing chamber 101 may include an integrated plasma source (not shown) that can generate plasma within the processing chamber. In either instance, the radical sensor 135 may be disposed within or connected to the processing chamber 101 rather than in or connected to the gas deliver lines 133 in embodiments.

Processing chamber 101 includes a substrate support assembly 150, according to some embodiments. Substrate support assembly 150 includes a puck 166 (e.g., may include an electrostatic chuck (ESC)). The puck 166 may perform chucking operations, e.g., vacuum chucking, electrostatic chucking, etc. Substrate support assembly 150 may further include base plate, cooling plate and/or insulator plate (not shown).

Processing chamber 100 includes chamber body 102 and lid 104 that enclose an interior volume 106. Chamber body 102 may be fabricated from aluminum, stainless steel, or other suitable material. Chamber body 102 generally includes sidewalls 108 and a bottom 110. An outer liner 116 may be disposed adjacent to side walls 108, e.g., to protect chamber body 102. Outer liner 116 may be fabricated and/or coated with a plasma or halogen-containing gas resistant material. Outer liner 116 may be fabricated from or coated with aluminum oxide. Outer liner 116 may be fabricated from or coated with yttria, yttrium alloy, oxides thereof, etc.

Exhaust port 126 may be defined in chamber body 102, and may couple interior volume 106 to a pump system 128. Pump system 128 may include one or more pumps, valves, lines, manifolds, tanks, etc., utilized to evacuate and regulate the pressure of interior volume 106.

Lid 104 may be supported on sidewall 108 of chamber body 102. Lid 104 may be openable, allowing access to interior volume 106. Lid 104 may provide a seal for processing chamber 100 when closed. Plasma source 158 may be coupled to processing chamber 100 to provide process, cleaning, backing, flushing, etc., gases and/or plasmas to interior volume 106 through gas distribution assembly 130. Gas distribution assembly 130 may be integrated with lid 104.

Examples of processing gases that may be used in processing chamber 100 include halogen-containing gases, such as F₂, C₂F₆, SF₆, SiCl₄, HBr, NF₃, CF₄, CHF₃, CH₂F₃, Cl₂ and SiF₄. Other reactive gases may include O₂ or N₂O. Non-reactive gases may be used for flushing or as carrier gases, such as N₂, He, Ar, etc. Gas distribution assembly 130 (e.g., showerhead) may include multiple apertures 132 on the downstream surface of gas distribution assembly 130. Apertures 132 may direct gas flow to the surface of substrate 144. In some embodiments, gas distribution assembly may include a nozzle (not pictured) extended through a hold in lid 104. A seal may be made between the nozzle and lid 104. Gas distribution assembly 130 may be fabricated and/or coated by a ceramic material, such as silicon carbide, yttrium oxide, etc., to provide resistance to processing conditions of processing chamber 100.

Substrate support assembly 150 is disposed in interior volume 106 of processing chamber 100 below gas distribution assembly 130. Substrate support assembly 150 holds a substrate 144 during processing. An inner liner (not shown) may be coated on the periphery of substrate support assembly 148. The inner liner 118 may share features (e.g., materials of manufacture, function, etc.) with outer liner 116.

Substrate support assembly 150 may include supporting pedestal 152, insulator plate, base plate, cooling plate, and puck 166. Puck 166 may include electrodes 536 for providing one or more functions. Electrodes 536 may include chucking electrodes (e.g., for securing substrate 144 to an upper surface of puck 166), heating electrodes, RF electrodes for plasma control, etc.

Protective ring 146 may be disposed over a portion of puck 166 at an outer perimeter of puck 166. Puck 166 may be coated with a protective layer (not shown). Protective layer 136 may be a ceramic such as Y₂O₃ (yttria or yttrium oxide), Y₄Al₂O₉ (YAM), Al₂O₃ (alumina), Y₃Al₅O₁₂ (YAG), YAlO₃ (YAP), quartz, SiC (silicon carbide), Si₃N₄ (silicon nitride), Sialon, Al (aluminum nitride), AlON (aluminum oxynitride), TiO₂ (titania), ZrO₂ (zirconia), TiC (titanium carbide), ZrC (zirconium carbide), TiN (titanium nitride), TiCN (titanium carbon nitride), Y₂O₃ stabilized ZrO₂ (YSZ), and so on. The protective layer may be a ceramic composite such as YAG distributed in an alumina matrix, a yttria-zirconia solid solution, a silicon carbide-silicon nitride solid solution, or the like. The protective layer may be sapphire or MgAlON.

Puck 166 may further include multiple gas passages such as grooves, mesas, and other features that may be formed in an upper surface of puck 166. Gas passages may be fluidly coupled to a gas source 105. Gas from gas source 105 may be utilized as a heat transfer or backside gas, may be utilized for control of one or more lift pins of puck 166, etc. Multiple gas sources may be utilized (not shown). Gas passages may provide a gas flow path for a backside gas such as He via holes drilled in puck 166. Backside gas may be provided at a controlled pressure into gas passages to enhance heat transfer between puck 166 and substrate 144.

Puck 166 may include one or more clamping electrodes. The clamping electrodes may be controlled by chucking power source 182. Clamping electrodes may further couple to one or more RF power sources through a matching circuit for maintaining a plasma formed from process and/or other gases within processing chamber 100. The RF power sources may be capable of producing an RF signal having a frequency from about 50 kilohertz (kHz) to about 3 gigahertz (GHz) and a power of up to about 10,000 Watts. Heating electrodes of puck 166 may be coupled to heater power source 178.

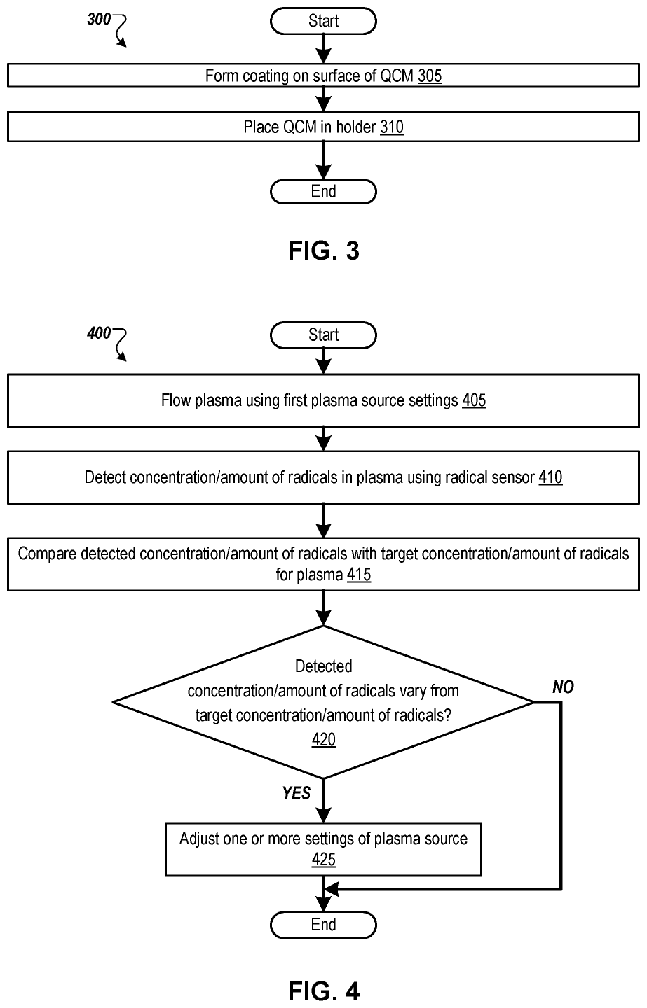

Controller 188 may control one or more parameters and/or set points of the plasma source 158 and/or processing chamber 101. System controller 188 can be and/or include a computing device such as a personal computer, a server computer, a programmable logic controller (PLC), a microcontroller, and so on. System controller 188 can include one or more processing devices, which can be general-purpose processing devices such as a microprocessor, central processing unit, or the like. More particularly, the processing device can be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device can also be one or more special-purpose processing devices such as an application specific integrated circuit (ASIC), a field programmable gate array (FPGA), a digital signal processor (DSP), network processor, or the like. System controller 188 can include a data storage device (e.g., one or more disk drives and/or solid state drives), a main memory, a static memory, a network interface, and/or other components. System controller 188 can execute instructions to perform any one or more of the methodologies and/or embodiments described herein. The instructions can be stored on a computer readable storage medium, which can include the main memory, static memory, secondary storage and/or processing device (during execution of the instructions). In embodiments, execution of the instructions by system controller 188 causes system controller to perform the methods of FIG. 4. For example, system controller 188 may receive measurements from radical sensor 135 indicating a concentration of a particular species of radicals in a received or generated plasma, and may adjust one or more properties or settings (e.g., such as a plasma power) of plasma source 158 responsive to the measured radical concentration. System controller 188 can also be configured to permit entry and display of data, operating commands, and the like by a human operator.

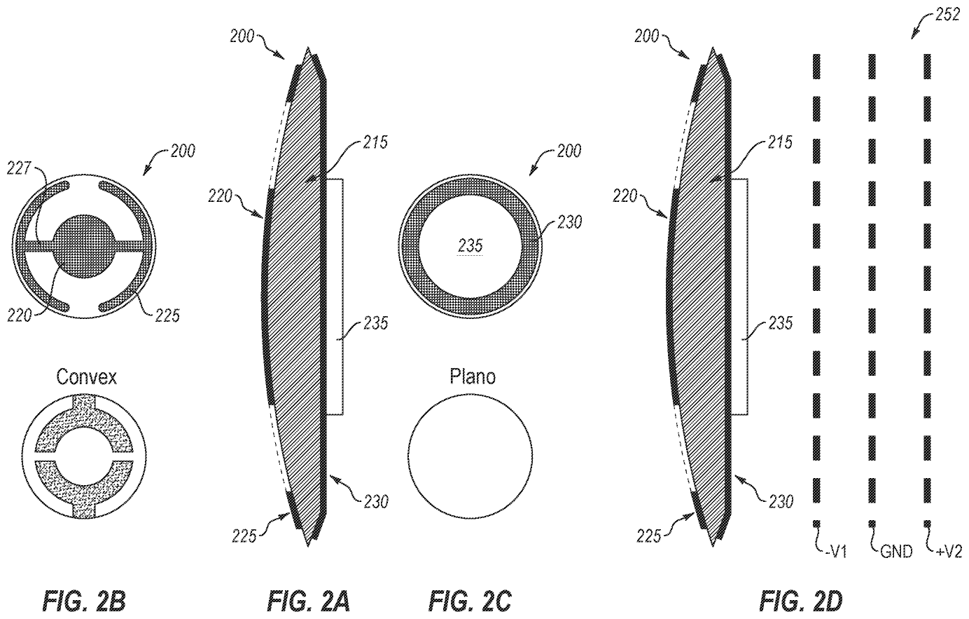

FIGS. 2A-2D illustrate embodiments of a sensor for detecting radicals of a target gas species, in accordance with embodiments of the present disclosure. FIG. 2A is a sectional side view of a radical sensor, according to some embodiments. FIG. 2B is a back view of the radical sensor of FIG. 2A, according to some embodiments. FIG. 2C is a front view of the radical sensor of FIG. 2A, according to some embodiments.

In embodiments, the radical sensor comprises a QCM sensor base. A piece of solid material of any shape can normally oscillate at certain resonant frequencies. By increasing the mass of the vibrating unit, the typical result is the decrease of that solid material's resonant frequencies. This is the basic principle of a QCM.

The QCM sensor base may include a thin plate of quartz crystal that oscillates in the thickness-shear mode because such a QCM sensor base has high sensitivity to mass change on the crystal. The piezoelectric nature of quartz crystal allows the crystal to be driven into oscillation and with its resonant frequency measured by simple electrical means. In embodiments, the quartz crystal is precisely cut at certain angles with respect to its crystallographic axes. In embodiments, the quartz crystal is an AT-cut quartz crystal.

As shown in FIGS. 2A-C, the radical sensor 200 includes a quartz crystal base 215, which may have a flat face and a convex face. The flat face may be a front face and the convex face may be a back face. The flat face may be covered by a front electrode 230. The convex face may be covered by a back electrode that includes a back electrode edge 225 connected to a back electrode center 220 via one or more electrode bridges 227. This configuration enables an alternating current to be applied to and/or read from the electrodes without compromising the ability of the quartz body 215 to oscillate freely. A sensing surface of the QCM may be a center region of the front face (e.g., a center region of front electrode 230). In embodiments, the sensing surface of the QCM is coated with a film 235 that is sensitive to reaction with a particular molecular species of a target gas species. The composition of the film 235 may depend on the application for which the radical sensor 200 will be used.

In some embodiments, the film 235 is composed of a material that reacts with a radical molecular species of a target gas, but that does not react to stable molecular species of the target gas. For example, the material may react to fluorine radicals, but may not react to stable molecules containing fluorine (e.g., F₂, C₂F₆, SF₆, NF₃, CF₄, CHF₃, CH₂F₃, etc.). The material may also not react to other molecules that may be included in a gas flow, whether those other molecules are radicals or stable molecular species. For example, the material may react to fluorine radicals, but may not react to carbon radicals, nitrogen radicals, hydrogen radicals, etc. Alternatively, the material may only react to hydrogen radicals, or may only react to carbon radicals, or may only react to some other radicals.

In one embodiment in which the radical sensor is tuned to detect fluorine radicals, the film or coating 235 comprises silicon dioxide (SiO₂), tungsten, or a tungsten oxide (e.g., tungsten (III) oxide or W₂O₃) and/or organic materials (such as photoresist). In one embodiment in which the radical sensor is tuned to detect fluorine radicals, the film or coating 235 comprises a transition metal that selectively reacts with fluorine radicals. In one embodiment in which the radical sensor is tuned to detect hydrogen radicals, the film or coating 235 comprises a polymer of carbon and hydrogen. One example of a polymer that may be used is polymethyl methacrylate (PMMA). In one embodiment in which the radical sensor is tuned to detect nitrogen radicals, the film or coating 235 comprises a fluorinated polymer. In embodiments, the target radicals react with the film 235 to form a gas, which consumes some portion of the film 235. The consumption of some portion of the film 235 reduces the number of molecules of the film 235, and thus reduces an overall mass of the film. This reduction in mass may be detected by the QCM sensor on which the film 235 has been formed.

In some embodiments, the reaction of the target radical species with the film 235 produces a solid byproduct. The solid byproduct adheres to the film 235, and thus increases a mass of the film 235. This increase in mass may be detected by the QCM sensor on which the film 235 has been formed.

In some embodiments, the reaction of the target radical species with the film 235 is an absorption process where the film 235 absorbs the target radical species. The absorption of the radical species causes a mass of the film 235 to increase. Once the film 235 becomes saturated with the target radical species and/or between process runs, a purge or cleaning process may be performed to cause the radical species to desorb from the film 235. In an example, a QCM with a coating of PMMA may be used to detect fluorine radicals. The PMMA may absorb fluorine radicals, and the mass change of the film caused by absorption of the fluorine radicals may be detected by a change in resonant frequency of the QCM. The fluorine radicals may then be desorbed by flowing another gas such as argon across the radical sensor 200.

In one embodiment, the film 235 has a thickness of about 1-100 microns. In one embodiment, the film 235 has a thickness of about 30-40 microns. Other thicknesses, such as 10, 20, 30, 40, 50, 60, 70, 80 or 90 microns may also be used for the film 235.

The QCM sensor base including the crystal 215 and electrodes 220, 225, 230 measures the Areal Mass Density (mass per unit area) of a material which uniformly covers the sensitive area on the sensing crystal. For heavy loading on the crystal 215, its accuracy depends on the knowledge of the shear-mode acoustic impedance value of the deposited material. Larger crystals do not have higher sensitivity. The QCM is not a weighing device because it does not require a gravitational force. It can be used in space with zero gravity. In embodiments, a thickness reading t_{f} may be derived from the areal mass density value, which is equivalent to t_{f}ρ_{f}, by using the density of the film pf. The entry of a wrong density value results a wrong thickness reading. The areal mass density measurement is in absolute value in embodiments. In embodiments, no calibration is needed for a properly designed QCM. Temperature variation, stress, gas adsorption and desorption, surface reaction, etc. can all give false signals.

The QCM can measure mass on a sensing surface of the QCM according to the equation m/A∝ρtⁿ, where m/A is mass per unit area, ρ is density of a material on a sensing surface of the QCM, t is thickness and n a constant (≥0) where for linear dependence n is equal to 1. In embodiments, the sensitivity of QCM can be down to better than 1×10⁻⁹ g/cm². In terms of thickness for a material, say, Al, with density ρ=2.7 g/cm³, this QCM sensitivity is equivalent to 0.1 Å of Al. Thickness change expressed in terms of mass per unit area, or areal mass density, is more appropriate at subatomic sizes.

A piezoelectric resonator can be represented by a simple equivalent circuit for electrical analysis, as shown in FIG. 2E. The mechanical behavior of a quartz crystal resonator (e.g., QCM) can be represented by an electrical equivalent model as shown. This is the so-called the Butterworth van Dyke (BVD) electrical model of a quartz crystal resonator. In the motional arm (the upper branch) of the BVD model, it consists of three components that determine the series resonance frequency of the quartz crystal plate. Ra corresponds to the energy dissipation due to mechanical coupling between the crystal and its holder. For QCM applications, added mass load on the crystal surface also causes Ra to increase. Lₐ corresponds to the mass being displaced during oscillation. For QCM applications, the total mass includes that of the crystal, the electrodes, and the deposited thin film materials. Cₐ corresponds to the stored energy in the oscillator that is related to the elastic properties of quartz, electrodes, and the deposited materials. The parasitic capacitance Co represents the total static capacitances of the crystal electrodes, the holder, and the connecting cable.

FIG. 2D is a sectional side view of the radical sensor of FIG. 2A with the addition of a charged grating or grid, according to some embodiments. The radical sensor 200 shown in FIGS. 2A-2C in embodiments measures all radicals without respect to charge. In some embodiments, it may be advantageous to measure only neutral radicals or only radicals having a particular charge. In order to measure only neutral radicals of a particular molecular species, a charged grating or grid 252 may be disposed in front of the front face of the radical sensor 200. The charged grating or grid 252 may include a stack of meshes (e.g., wire meshes) that have particular charge. In one embodiment, as shown, a first grating or mesh is positively charged, a second grating or mesh may be grounded, and a third grating or mesh may be negatively charged. The positively charged mesh or grating may repel positively charged molecules or ions and the negatively charged mesh or grating may repel negatively charged molecules or ions. As a result, the only molecules that reach the film 235 may be neutral molecules. Of the neutral molecules that reach the film 235, only the radicals of a particular gas species may actually react with the film 235.

In one embodiment, in order to measure an amount of positively and/or negatively charged radicals, a pair of radical sensors may be used. A first radical sensor may include the charged gratings or grids, and a second radical sensor may not include the charged gratings or grids. All radicals of a target gas species may be detected by the second radical sensor, and only neutral radicals of the target gas species may be detected by the first radical sensor. A difference between the measurements of the two radical sensors may then be computed to determine an amount of the radicals detected by the second radical sensor that were attributable to charged radicals. The grating may be modified to only filter out positively charged molecules/ions or to only filter out negatively charged molecules. Accordingly, by combining two or more radical sensors, each with a different grating configuration (e.g., one not including any grating), an amount of positively charged radicals may be detected, an amount of negatively charged radicals may be detected, and/or an amount of neutral radicals may be detected.

FIG. 2F shows an expanded view of one example of a radical sensor included in a sensor holder. As shown, a cover compresses a quartz crystal (e.g., having the form shown in FIGS. 2A-D, with a coating that is reactive only to a specific radical species on a sensing surface) against a spring contact. An aperture exposes the sensing surface that includes the coating. Many other different configurations of sensor holders may also be used, such as is shown in FIGS. 2G and 2H.

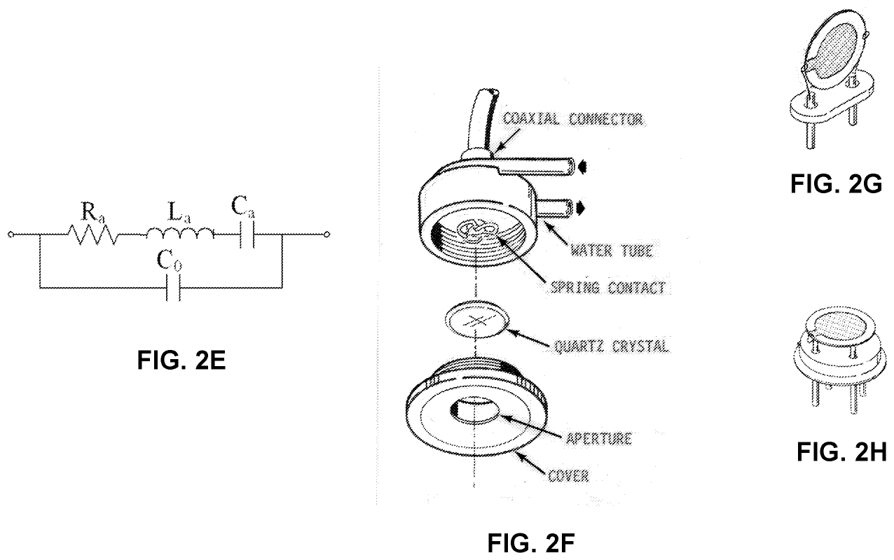

FIG. 3 is a flow chart of one embodiment of a method 300 for manufacturing a radical sensor. At block 305 of method 300, a coating is formed on a sensing surface of a QCM or other piezoelectric substrate. The coating may be composed of a material that is selectively reactive to radicals of a target gas species, but that is not reactive to stable molecules of the particular gas species, and that is not reactive to stable molecules or radicals of other gas species that will be used together with the target gas species. The sensing surface may be coated by first placing a hard or soft mask over the a face of the QCM or other piezoelectric substrate. The exposed region of the face of the QCM or other piezoelectric substrate may then be coated, and the mask may be removed. Alternatively, the coating may be formed on an entirety of a surface, and a portion of the coating may then be selectively removed (e.g., by forming a hard or soft mask over the portion of coating that is not to be removed, and then etching the exposed portion of the coating, and finally removing the mask). At block 310, the radical sensor is then placed in a sensor holder, such as any of those discussed above.

FIG. 4 is a flow chart of one embodiment for a method 400 of controlling a plasma source using a radical sensor. At block 405 of method 400, a manufacturing system flows a plasma using first plasma source settings. The plasma may be generated by a remote plasma source (e.g., a plasma source external to a process chamber) or by a local plasma source (e.g., a plasma source internal to a process chamber). At block 410, a radical sensor (e.g., as described hereinabove) is used to detect a concentration/amount of radicals in the plasma. The radicals of a target gas species may react with a coating on the radical sensor, causing a mass on a sensing surface of the radical sensor to change. The density and/or mass of the coating may be known, and a change in the mass of the coating may be detected based on a change in the resonant frequency of an oscillating piezoelectric material (e.g., QCM) of the radical sensor. This change in mass together with knowledge about the mass of the materials that make up the coating may be used to determine a number of radicals that reacted with the coating, and thus the concentration and/or amount of radicals in the gas flow.

At block 415, processing logic compares the detected concentration/amount of radicals of the target gas species to a target concentration/amount of radicals for the plasma. At block 420, processing logic determines whether the detected concentration/amount of radicals of the target gas species varies from the target concentration/amount by more than a threshold amount (e.g., if a difference between the target concentration and the detected concentration is more than a difference threshold). If the difference exceeds a difference threshold, the method continues to block 425 and one or more settings of the plasma source are adjusted. For example, the plasma power may be increased in increase an amount of radicals that are included in the plasma or may be decreased to reduce an amount of radicals that are included in the plasma. If the difference is less than the difference threshold, then the method may end.

Unless specifically stated otherwise, terms such as “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may include a general purpose system selectively configured to perform methods described herein.

The terms “over,” “under,” “between,” “disposed on,” “support,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

1. A sensor device comprising:
a quartz crystal microbalance (QCM); and
a coating on at least a portion of a surface of the QCM, wherein the coating selectively reacts with radicals of a target gas but does not react with stable molecules of the target gas;
wherein the QCM is configured such that a resonant frequency of the QCM changes in response to reaction of the radicals of the target gas to the coating, and wherein the change in the resonant frequency of the QCM correlates to an amount of the radicals of the target gas that has reacted with the coating.

2. The sensor device of claim 1, wherein the coating comprises a material that reacts with the radicals of the target gas to form a gaseous byproduct and reduce a thickness of the coating.

3. The sensor device of claim 2, wherein the target gas comprises hydrogen, and wherein the material comprises a polymer of carbon and hydrogen.

4. The sensor device of claim 3, wherein the material comprises polymethyl methacrylate (PMMA).

5. The sensor device of claim 2, wherein the target gas comprises fluorine, and wherein the material comprises silicon dioxide (SiO₂), tungsten, or an oxide of tungsten.

6. The sensor device of claim 5, wherein the material comprises tungsten(III) oxide (W₂O₃).

7. The sensor device of claim 2, wherein the target gas comprises nitrogen, and wherein the material comprises a fluorinated polymer.

8. The sensor device of claim 1, wherein the coating comprises a material that reacts with the radicals of the target gas to form a solid byproduct and increase a thickness of the coating.

9. The sensor device of claim 1, wherein the coating comprises a material that absorbs the radicals of the target gas to increase a mass of the coating.

10. The sensor device of claim 9, wherein responsive to application of a second gas to the sensor device the radicals of the target gas desorb from the material.

11. The sensor device of claim 1, wherein the target gas is a constituent of a gas flow comprising a plurality of gases, and wherein the coating does not react to radicals of any gases of the plurality of gases other than the radicals of the target gas.

12. The sensor device of claim 1, wherein the coating has a thickness of 1-100 microns.

13. The sensor device of claim 1, wherein the surface of the QCM comprising the coating corresponds to a front electrode of the QCM.

14. The sensor device of claim 1, further comprising:
a charged grid over the coating, wherein the charged grid repels ions of the target gas such that only neutral radicals of the target gas reach the coating.

15. A manufacturing system, comprising:
a plasma source to generate a plasma;
a process chamber connected to the plasma source via one or more delivery lines; and
a sensor device connected to at least one of the plasma source, the process chamber or the one or more delivery lines, wherein the sensor device comprises a quartz crystal microbalance (QCM) comprising a coating that selectively reacts with radicals of a target gas and does not react to stable molecules of the target gas to measure an amount of the radicals of the target gas.

16. The manufacturing system of claim 15, wherein the QCM is configured such that a resonant frequency of the QCM changes in response to reaction of the radicals of the target gas to the coating, and wherein the change in the resonant frequency of the QCM correlates to an amount of the radicals of the target gas that have reacted with the coating.

17. The manufacturing system of claim 15, further comprising:
a controller, connected to the sensor device and to the plasma source, wherein the controller is to adjust one or more parameters of the plasma source responsive to the amount of radicals of the target gas detected by the sensor device.

18. The manufacturing system of claim 17, wherein the controller is to increase a plasma power responsive to a determination that the amount of radicals of the target gas is below a target threshold.

19. A method comprising:
receiving a gas flow comprising one or more gases, the one or more gases comprising a first plurality of stable molecules of a target gas and a second plurality of radicals of the target gas; and
measuring the second plurality of radicals of the target gas without measuring the first plurality of stable molecules of the target gas using a quartz crystal microbalance (QCM) comprising a coating on at least one surface that reacts with the second plurality of radicals of the target gas but not with the first plurality of stable molecules of the target gas.

20. The method of claim 19, wherein the one or more gases comprise a plasma output by a remote plasma source, the method further comprising:
adjusting one or more parameters of the remote plasma source to adjust a concentration of radicals of the target gas responsive to measuring the second plurality of radicals of the target gas.
